(19) United States
(12) Patent Application Publication
(10) Pub. No.: US 2007/0093750 A1
(43) Pub. Date: Apr. 26, 2007

(54) Insulin pump

(76) Inventors: Kil-Wan Jan, Seoul (KR); Yong-Taeg Nam, Kyunggi-do (KR); Chi-Hwan Moon, Inchon (KR)

Correspondence Address: Dilworth & Barrese, 333 Earle Ovington Blvd, Suite 702, Uniondale, NY, 11553, US

(21) Appl. No.: 10/550,811

(22) PCT Filed: Mar. 25, 2004
(86) PCT No.: PCT/KR04/00662

§ 371 Date: Jul. 27, 2006

(30) Foreign Application Priority Data
Mar. 27, 2003 (KR) 10-2003-0019117

(51) Int. Cl. A61M 37/00

(52) U.S. Cl. 604/131

(57) ABSTRACT

The present relates to an automatic insulin pump, which allows the rotary shaft only to rotate piston advancing means to remove vertical load applied to a rotary shaft, thereby improving durability thereof. The insulin pump includes: an injector having a syringe for containing insulin therein, and a piston inserted into the rear end of the syringe for providing the syringe with insulin discharge pressure; a housing having an injector receiving space formed in an appropriate position thereof, the injector receiving space having a partition wall formed at the rear end thereof; a rotary shaft having a non-circular section and a predetermined length; power supply means for rotating the rotary shaft at a predetermined speed; a push plate assembly for providing the piston with ahead power by pushing the piston, the push plate assembly having a disk part having a male screw formed on the outer circumferential surface thereof and a coupling hole axially coupled with the rotary shaft at the central portion thereof to allow for forward and backward movement of the rotary shaft, which passes through the coupling hole; and a hollow cylindrical type push plate case inserted into the injector receiving space of the rear end of the syringe, for the piston to pass therethrough, the push plate case having a female screw formed on the inner circumferential surface thereof to be coupled with the male screw of the disk part for allowing the disk part to carry out a spirally forward and backward movement.

### TECHNICAL FIELD

The present invention relates to an automatic insulin pump, and more particularly, to an automatic insulin pump, which can reduce load applied to a rotary shaft providing ahead power to a piston to thereby improve durability thereof.

### BACKGROUND ART

In general, in a parenteral therapy for a glycosuria patient, the glycosuria patient or a protector for the glycosuria patient injects insulin into the patient twice or three times a day. At this time, the amount of insulin needed for the patient varies according to time and patient's body conditions. For example, more insulin needs to be injected during taking a meal than in normal times. However, the conventional method for manually injecting insulin has several disadvantages in that it is impossible to inject an exact amount of insulin to the patient, and in that the patient suffers from an inconvenience of going to a hospital to administer insulin more than insulin injected in normal times.

To solve the above disadvantages, Korean Patent No. 290253 (patented on Feb. 28, 2001) discloses an “insulin pump” capable of automatically injecting a fixed amount of insulin every certain time by driving a piston in a state where an injection needle is always inserted into a body fat region of the patient's abdomen part.

FIG. 1 is a front view of a structure of a conventional automatic insulin pump, FIG. 2 is a plan view of the automatic insulin pump of FIG. 1, FIG. 3 is a view showing a piston driving mechanism of the automatic insulin pump, and FIG. 4 is a perspective view showing a used condition of the automatic insulin pump of FIG. 1. As shown in FIGS. 1 to 4, an injector is mounted at a side of a box type housing 20 of a conventional automatic insulin pump in a longitudinal direction, and a push member 50 is mounted at the lower portion of the injector to drive the injector. The injector includes a cylindrical syringe 21 for containing insulin therein and a piston 22 inserted into the syringe 21 for pushing the insulin through a tube 1. A disk type push member 50 is mounted on the lower end of the piston 22, and a female screw (not shown in the drawings) is formed at the center of the push member 50.

Meanwhile, a motor (not shown) and a power supply means 30, which has a number of deceleration gear lines (not shown) for decelerating a rotational speed of the motor, are mounted on the lower portion of the housing 20, and a rotary shaft 31 is mounted on the final gear of the deceleration gear lines. The rotary shaft 31 has a male screw of the circumferential surface thereof, and the male screw is coupled with the female screw of the push member 50. As a result, the push member 50 advances according to the rotation of the rotary shaft 31, the piston 22 advances inside the syringe 21, and thereby, the insulin corresponding to an advanced amount of the piston 22 is injected into the patient's body through the tube 1 and an injection needle 3. In the drawings, unexplained reference numeral 10 designates a cover for allowing the injector to be drawn to the outside of the housing 20 when the insulin is filled up, 2 designates a connector for connecting the tube 1 to the syringe 21, and 40 designates a sealing cap for preventing penetration of moisture into the power supply means 30.

However, the conventional automatic insulin pump has a problem in that lots of vertical load is applied to the rotary shaft as the rotary shaft is directed connected to the piston to drive the piston, and thereby, durability of the rotary shaft is deteriorated. Furthermore, the conventional automatic insulin pump has another problem in that a contact area between the male screw of the rotary shaft and the female screw of the push member is relatively reduced as the diameter of the rotary shaft becomes small, and thereby, the piston cannot carry out a stable and smooth advancing motion.

### DISCLOSURE OF INVENTION

Accordingly, the present invention has been made in view of the above problems, and it is an object of the present invention to provide an automatic insulin pump, which allows the rotary shaft to only serve rotate piston advancing means (push plate assembly) to remove vertical load applied to a rotary shaft, thereby improving durability thereof.

To achieve the above object, the present invention provides an automatic insulin pump including: an injector having a syringe for containing insulin therein, and a piston inserted into the rear end of the syringe for providing the syringe with insulin discharge pressure; a housing having an injector receiving space formed in an appropriate position thereof, the injector receiving space having a partition wall formed at the rear end thereof; a rotary shaft having a non-circular section and a predetermined length; power supply means for rotating the rotary shaft at a predetermined speed; a push plate assembly for providing the piston with ahead power by pushing the piston, the push plate assembly having a disk part having a male screw formed on the outer circumferential surface thereof and a coupling hole axially coupled with the rotary shaft at the central portion thereof to allow for forward and backward movement of the rotary shaft, which passes through the coupling hole; and a hollow cylindrical type push plate case inserted into the injector receiving space of the rear end of the syringe, for the piston to pass therethrough, the push plate case having a female screw formed on the inner circumferential surface thereof to be coupled with the male screw of the disk part for allowing the disk part to carry out a spirally forward and backward movement.

### BRIEF DESCRIPTION OF DRAWINGS

Further objects and advantages of the invention can be more fully understood from the following detailed description taken in conjunction with the accompanying drawings in which:

FIG. 1 is a front view of a structure of a conventional automatic insulin pump;

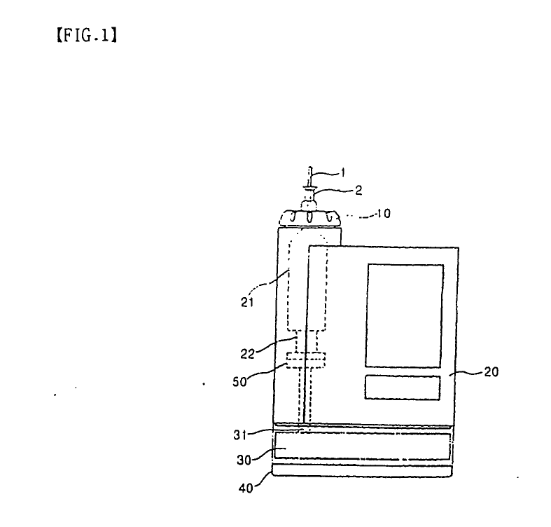

FIG. 2 is a plan view of the automatic insulin pump of FIG. 1;

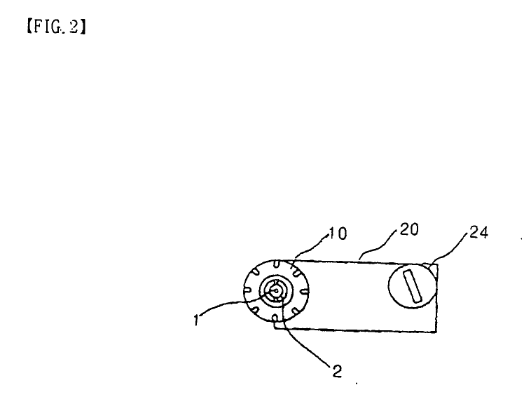

FIG. 3 is a view showing a piston driving mechanism of the automatic insulin pump;

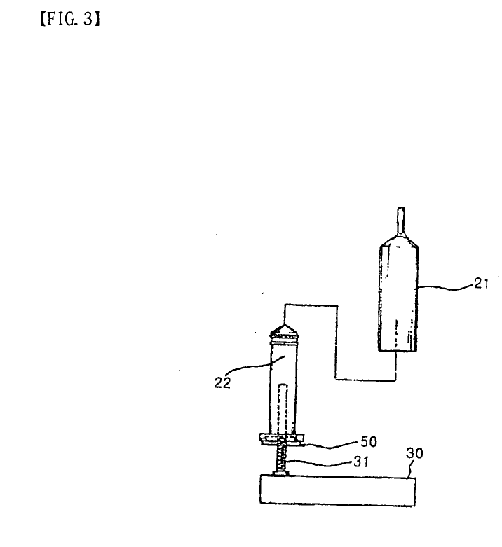

FIG. 4 is a perspective view showing a used condition of the automatic insulin pump of FIG. 1;

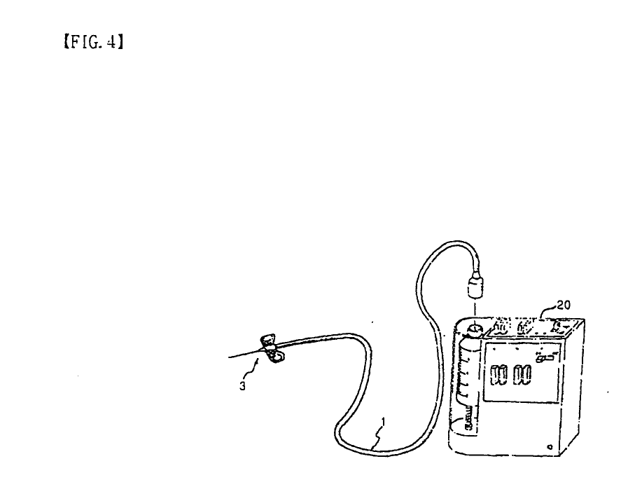

FIG. 5 is a generally perspective view of an automatic insulin pump according to a preferred embodiment of the present invention;

FIG. 6 is an exploded perspective view of the automatic insulin pump of the present invention;

FIG. 7 is a sectional view showing an injector driving mechanism of the automatic insulin pump of the present invention;

FIG. 8 is a bottom view of a push plate assembly of the automatic insulin pump of the present invention;

FIG. 9 is a perspective view of an auxiliary coupling tool of the automatic insulin pump of the present invention; and

FIG. 10 is a perspective view showing a used condition of the automatic insulin pump of the present invention.

### BEST MODE FOR CARRYING OUT THE INVENTION

The present invention will now be described in detail in connection with preferred embodiments with reference to the accompanying drawings.

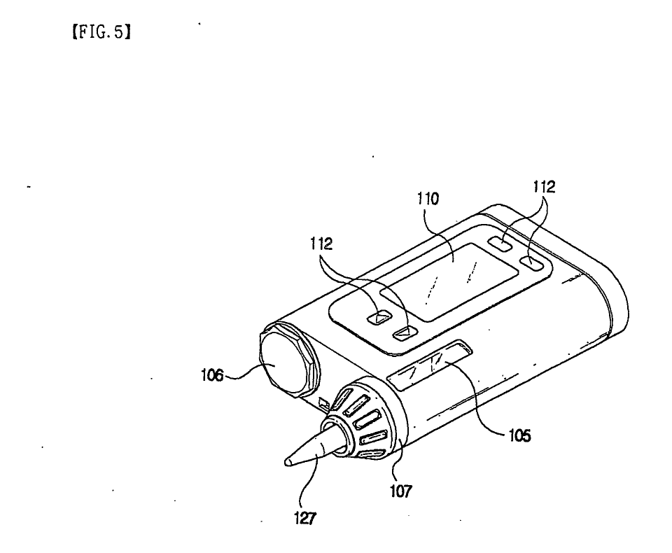

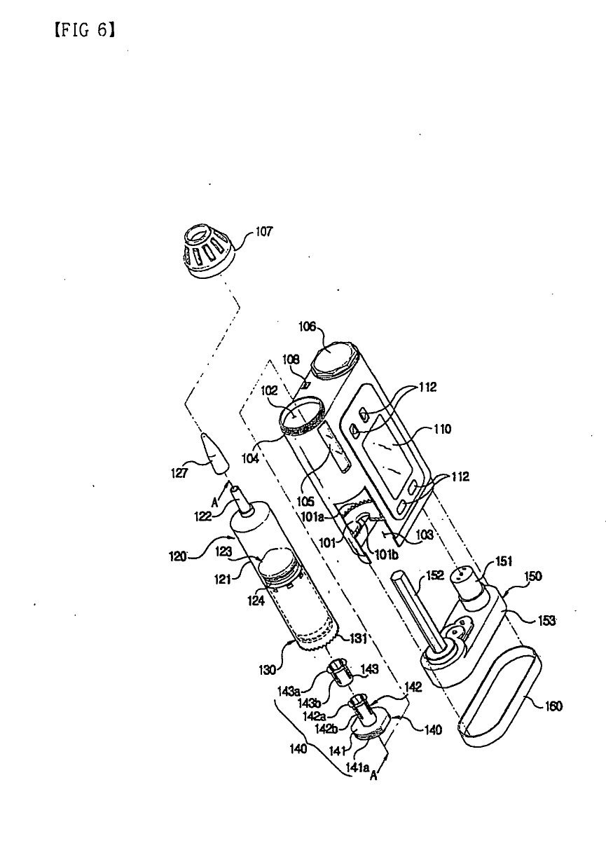

FIG. 5 is a generally perspective view of an automatic insulin pump according to a preferred embodiment of the present invention, and FIG. 6 is an exploded perspective view of the automatic insulin pump of the present invention. As shown in FIGS. 5 and 6, the automatic insulin pump according to the present invention has an injector 120 contained inside a small box type housing 100. For this, the housing 100 has a cylindrical injector receiving space 102 formed at a side of the inside of the housing 100 in a longitudinal direction. The housing 100 has a battery space (not shown) formed at an appropriate position thereof for supplying electric power to various electric components. The reference numeral 106 designates a battery cover for restricting a battery (not shown) inside the battery space. The housing 100 also includes an LCD window 110 mounted on the front surface thereof for displaying various driving conditions of the insulin pump, and a number of buttons 112 arranged on both sides of the LCD window 110.

The housing 100 also has a receiving space formed at the rear end portion thereof for receiving power supply means 150 providing driving power for discharging insulin to the injector 120. The power supply means 150 includes a case 153, a DC motor 151 mounted at an appropriate position of the case 153, a number of deceleration gear lines (not shown) mounted inside the case 153 for decelerating rotational frequency of the DC motor 151, and a hexagonal rotary shaft connected to the final end of the deceleration gear lines and exposed to the outside of the case 153 to a predetermined length. The reference numeral 160 designates a sealing cap for preventing penetration of moisture into cracks unavoidably formed in the case 153, 108 designates a line holding hole for connecting a line for allowing the patient to hang the line on the patient's neck.

Meanwhile, a male screw 104 of a predetermined length is formed on the front end of the housing restricting the injector receiving space 102, and a separation cap 107 is crewed to the male screw 104 for separating the injector 120 from the housing 100 if needed. For the separation of the injector 120, the separation cap 107 has a female screw (not shown) formed on the inner circumferential surface thereof, and the female screw is coupled with the male screw of the housing 100. The injector receiving space 102 has a partition wall 101 formed at the rear end thereof, at least one or more idle rotation preventing saws 110a formed on the circumferential surface of the partition wall 101, and a rotary shaft hole 101b formed at the central portion of the partition wall 101 for passing the hexagonal rotary shaft thereinto.

Meanwhile, the injector 120 includes a cylindrical syringe 121 for containing the insulin and having a soft tube connector 122 formed at the front end of the syringe 121, and a cylindrical hollow piston 123 inserted through the rear end of the syringe 121 for discharging the insulin toward the soft tube connector 122. The front end of the piston 123 is closed, an O-ring 124 is mounted around the closed front end of the piston 123, and the rear end of the piston 123 is opened. The reference numeral 127 designates a sealing cap for preventing leakage of insulin through the soft tube connector 122 when the insulin is filled up or needed, and 105 designates a syringe window formed on the wall of the injector receiving space 102 for checking an amount of the insulin contained in the syringe 121.

Inside the injector receiving space 102, a push plate case 130 for rotatably supporting a push plate assembly 140 moving forwardly (or backwardly) the piston 123 is located in a state in which the push plate case 130 is in contact with the syringe 121. Idle rotation preventing saws 131, which engage with the idle rotation preventing saws 101a formed on the partition wall 101, are formed at the rear end of the push plate case 130. As a result, when the separation cap 107 is coupled with the male screw 104 in a state in which the injector 120 and the push plate case 130 are contained in the injector receiving space 102, the front end of the syringe 121 is restricted by the separation cap 107, and the rear end of the push plate case 130 contacting with the rear end of the syringe 121 is restricted by the partition wall 101, so that the forward and backward movement of the syringe 121 inside the injector receiving space 102 can be restricted. Moreover, the idle rotation of the push plate case 130 can be restricted by the above structure. Meanwhile, the push plate case 130 has a female screw 132 formed on the inner circumferential surface thereof to a predetermined length (the entire length of the inner circumference of the push plate case 130 in this embodiment).

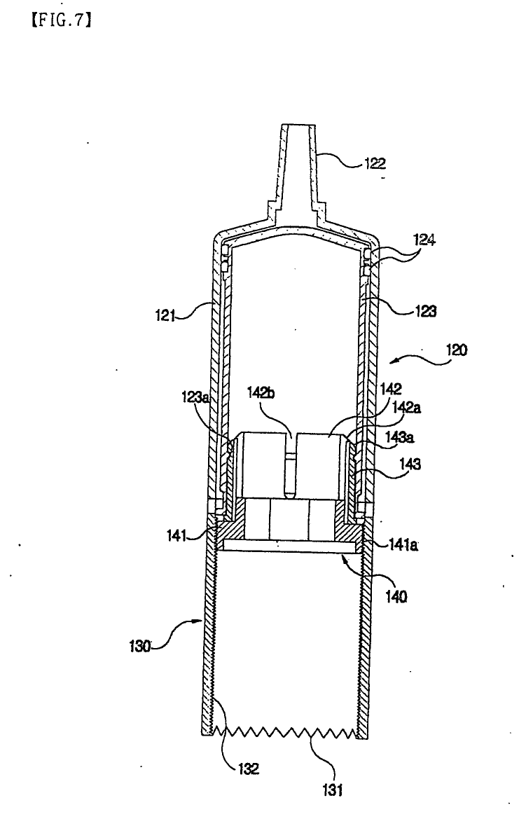

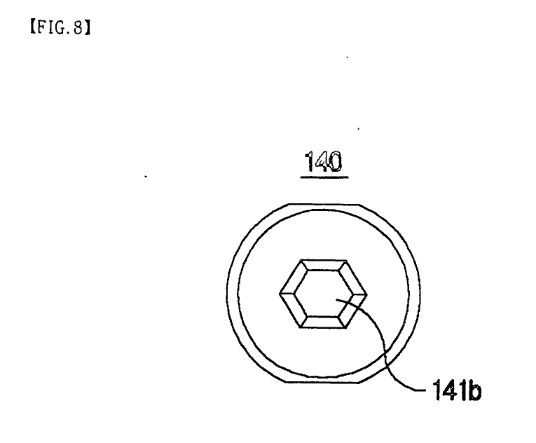

FIG. 7 is a sectional view showing an injector driving mechanism of the automatic insulin pump of the present invention, and FIG. 8 is a bottom view of a push plate assembly of the automatic insulin pump of the present invention. As shown in FIGS. 7 and 8, the push plate assembly 140 includes: a disk part 141 having a male screw 141a formed on the circumferential surface thereof to be coupled with the female screw 132 formed on the inner circumferential surface of the push plate case 130; a cylindrical type idle rotation sleeve 142 protruding from the disk surface of the disk part 141 to a predetermined length; and a cylindrical type fixed sleeve 143 inserted and fixed into the inner circumferential surface of the rear end portion of the piston in a state in which it is inserted into the inner circumferential surface of the idle rotation sleeve 142 so as to rotatably support the idle rotation sleeve 142. In the drawings, the reference numeral 142b designates a hole for allowing diameter contraction of the idle rotation sleeve so that the fixed sleeve 143 can be smoothly inserted into the idle rotation sleeve 142, and 142a designates a locking protrusion for preventing an easy separation of the inserted fixed sleeve 143 from the piston 123. For this, the piston 123 has a locking protrusion 123a formed on the inner circumferential surface of the rear portion thereof for preventing separation of the locking protrusion 143a. In the above structure, if a difference between the outer diameter of the idle rotation sleeve 142 and the inner diameter of the fixed sleeve 143 is larger than a difference between the outer diameter of the fixed sleeve 143 and the inner diameter of the piston 123, the idle rotation sleeve 142 can be freely rotated, i.e., idly rotated, inside the fixed sleeve 143 fixed to the piston 123.

Meanwhile, the disk part 141 has a hexagonal coupling hole 141b formed at the center thereof to be axially coupled with the hexagonal rotary shaft 152. The hexagonal rotary shaft 152 is coupled with the coupling hole 141b not in an idly rotating way but in a forwardly or backwardly moving way. The above coupling structure can be achieved, for example, by making the diameter of the coupling hole 141b(length of opposed apexes) larger than the diameter of the hexagonal rotary shaft 152 (length of opposed apexes). By the above structure, only rotation power for rotating only the coupling hole 141b is applied to the hexagonal rotary shaft 152 without applying any other vertical load.

As a result, when the hexagonal rotary shaft 152 is rotated together with the rotation of the motor 151, the disk part 141 of the push plate assembly 140 is also rotated. In this process, the male screw 141a formed on the disk part 141 is rotated along the screw thread of the female screw 132 formed on the inner circumferential surface of the push plate case 130, and advances to a predetermine length per 1 rpm, thereby pushing the piston 123.

However, in this process, as the piston 123 is restricted to the fixed sleeve 143 by a Predetermined power and the idle rotation sleeve 142 idly rotates inside the fixed sleeve 142, the piston 123 can advance smoothly without any influence by rotational force.

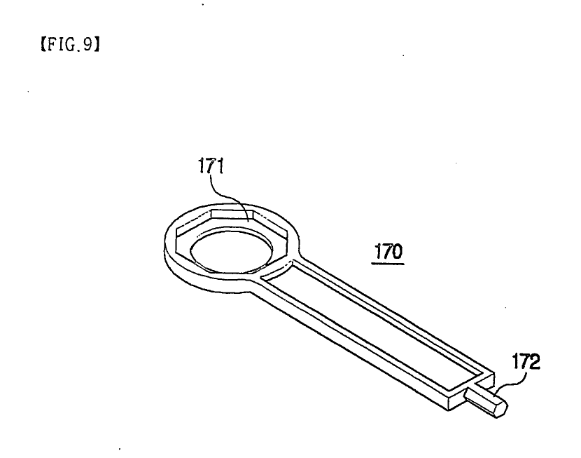

FIG. 9 is a perspective view of an auxiliary coupling tool of the automatic insulin pump of the present invention. When the automatic insulin pump according to the present invention is sold, an auxiliary coupling tool 170 shown in

FIG. 9 is provided. The auxiliary coupling tool 170 includes a hexagonal wrench 172 coupled to the coupling hole 141b for manually moving the push plate assembly 140, i.e., the piston 123 in forward and backward directions, and a hexagonal receptacle 171 formed integrally with the hexagonal wrench 172 for opening and closing the hexagonal battery cap 106.

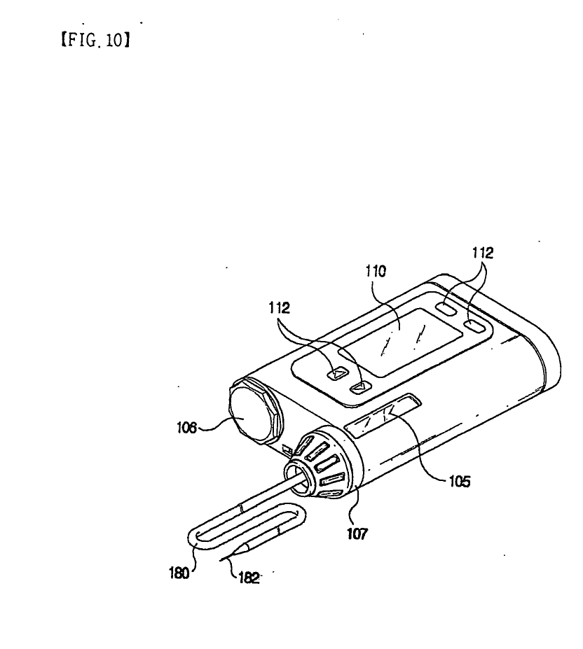

FIG. 10 is a perspective view showing a used condition of the automatic insulin pump of the present invention. As shown in FIG. 10, to fill up the insulin into the injector 120, first, after the separation cap 107 is opened and the push plate assembly 140 is assembled to the injector 120, an injection needle is stuck into the soft tube connector 122 in a condition in which the injector 120 is separated, and the piston 123 is pulled out so that the insulin of a desired amount is contained into the syringe 121 from an insulin tank. Next, the push plate assembly 140 is manually advanced by the wrench 172 of the auxiliary coupling tool 170 so that air bubbles contained inside the syringe 121 are taken out. After that, in a condition in which the sealing cap 127 closes the soft tube connector 122, the injector 120 is inserted into the injector receiving space 102, and the separation cap 107 is closed. In this condition, if the patient wants to use the insulin pump, the patient removes the sealing cap 127 from the soft tube connector 122, and connects a soft tube 180, which has the injection needle 182 at an end thereof, to the soft tube connector 122. After that, the patient injects the insulin into the soft tube 180 by pressing an operation button 112 so as to completely remove the air bubbles from the soft tube 180, and then, inserts the injection needle 182 into the body fat layer of the abdomen part.

While the present invention has been described with reference to the particular illustrative embodiment, it is not to be restricted by the embodiment but only by the appended claims. It is to be appreciated that those skilled in the art can change or modify the embodiments without departing from the scope and spirit of the present invention. In the above embodiment of the present invention, the rotary shaft and the coupling hole are in the form of a hexagon, but may be in the form of one of lots of polygons, or in one of other forms.

### INDUSTRIAL APPLICABILITY

As described above, the automatic insulin pump according to the present invention allows the rotary shaft to only serve rotate push plate assembly to remove vertical load applied to a rotary shaft, thereby improving durability thereof.

Furthermore, the present invention can disperse power by contacting the push plate assembly and the push plate case in a wide area for advancing the piston, thereby advancing the piston smoothly and stably. In addition, the present invention can remove the external force applied to the piston as spirally ahead power generated from the disk part of the push plate is provided to the piston only as ahead power, thereby improving durability of the piston.

1. An automatic insulin pump comprising:
an injector having a syringe for containing insulin therein, and a piston inserted into the rear end of the syringe for providing the syringe with insulin discharge pressure;
a housing having an injector receiving space formed in an appropriate position thereof, the injector receiving space having a partition wall formed at the rear end thereof;
a rotary shaft having a non-circular section and a predetermined length;
a power supply means for rotating the rotary shaft at a predetermined speed;
a push plate assembly for providing the piston with ahead power by pushing the piston, the push plate assembly having a disk part having a male screw formed on the outer circumferential surface thereof and a coupling hole axially coupled with the rotary shaft at the central portion thereof to allow for forward and backward movement of the rotary shaft, which passes through the coupling hole; and
a hollow cylindrical type push plate case inserted into the injector receiving space of the rear end of the syringe, for the piston to pass therethrough, the push plate case having a female screw formed on the inner circumferential surface thereof to be coupled with the male screw of the disk part for allowing the disk part to carry out a spirally forward and backward movement.

2. The automatic insulin pump according to claim 1, wherein the front end of the injector receiving space restricts the front end of the syringe and is limited by a separable cap, and the rear end of the injector receiving space is limited by the partition wall restricting the rear end of the push plate case, and
wherein the partition wall has idle-rotation preventing saws, and the push plate case has idle-rotation preventing saws formed at the rear end thereof and engaging with the idle-rotation saws of the partition wall.

3. The automatic insulin pump according to claim 1, wherein the piston has a hollow cylindrical body having the closed front end and the opened rear end, and
wherein the push plate assembly includes: the disk part, a hollow cylindrical type idle rotation sleeve protruding from the surface of a side of the disk part; and a hollow cylindrical type fixed sleeve inserted into the outer circumferential surface of the idle rotation sleeve to carry out the idle rotation, the outer circumferential surface of the fixed sleeve being fixed to the inner circumferential surface of the rear end portion of the piston.

4. The automatic insulin pump according to claim 3, wherein the idle rotation sleeve has a hole for allowing for diameter contraction of the idle rotation sleeve and a locking protrusion for preventing separation of the idle rotation sleeve, and the fixed sleeve has a hole for allowing for diameter contraction of the fixed sleeve and a locking protrusion for preventing separation of the fixed sleeve.

5. The automatic insulin pump according to claim 3, wherein the housing has string holding means formed at a proper position thereof to allow a patient to hang a string on his or her neck.

6. The automatic insulin pump according to claim 1, further comprising a wrench for allowing for a manual operation of the coupling hole.
